(19) United States
(12) Patent Application Publication
(10) Pub. No.: US 2012/0046694 A1
(43) Pub. Date: Feb. 23, 2012

(54) Device for Applying Algorithmic Afferent Modulation and Method of Use

(76) Inventor: Edward W. Miller, Salem, OR (US)

(21) Appl. No.: 12/861,763

(22) Filed: Aug. 23, 2010

(51) Int. Cl. A61F 5/00

(52) U.S. Cl. 606/239

(57) ABSTRACT

Disclosed is a chiropractic instrument capable of achieving the benefits of algorithmic pre-stretch, power-stretch, and recoil and release in reciprocating therapy to human tissue. In particular, that application teaches a plunging-probing technique wherein a barrage of power-stretch impulses is added to a time-modulated ramp of increasing and decreasing stretches, while offering an inherent safety-limit which prevents, upon operator error force, beyond the max given on its label, while providing meaningful tactile feedback to the practitioner. The new idea yields tethered reproducible power-stretches which only occur concurrently with achieving precise, variable, and preset levels of tissue pre-stretch force.

### BACKGROUND OF THE INVENTION

The present invention pertains generally to a chiropractic instrument, but more particularly, is focused on providing an algorithmic afferent modulation instrument that consistently replicates a chiropractic afferent-work-algorithm, while producing up to 200% the work afferent of prior art.

The advent of the simple ballistic hammer-to-the-anvil thrusting device to provide chiropractic therapy has benefited both patient and practitioner. It offered a directed force hand-saving alternative to traditional hand manipulative treatment, which can be physically stressful and debilitating to the chiropractor with long term practice. Ultimately, research on mechanoreceptors displacement therapy has led to another paradigm, a better understanding of the needs of afferent communication and the technology that serves it.

A mechanoreceptor is a sensory organ receptor that responds to mechanical pressure or distortion/deformation—by producing an action efferent to the brain. A variety of mechanoreceptors exist in the joints, ligaments, disc; muscles; skin etc. Different types of sensory end organs respond at different thresholds—and to different types of mechanical stimuli such as: a routine of stretch, power-stretch and release, compression, resonance, etc. Countless specialized nerve endings are present throughout the soft tissues of the musculoskeletal system which interact with the central nervous system and coordinate our body movements, our postural alignment, and our balance. When there is a communications breakdown, or when improper information is supplied by one or more of these sensors, efficiency of movement decreases. Often this leads chiropractors to utilize, increasing and sometimes, excessive levels of force. This can be harmful and possibly injurious to the muscles and joints, resulting in problems with postural coordination and/or joint alignment. Beyond being just an annoyance, faulty coordination or misalignments can also be the source of chronic, unresolved pain. An effective afferent-stimulation device is needed to achieve successful tissue-release with lower levels of force.

Location of Nerve Endings (in the muscles): The most important sensory nerve endings for controlling the muscular system are the muscle spindle fibers and the Golgi tendon organs. Muscle spindle fibers are found interspersed within the contractile fibers of all skeletal muscles, with the highest concentration in the central portion (belly) of each muscle. Muscle spindles respond to changes in the length of muscles. A complex circuitry of these nerve endings, with interconnections in the dorsal horn of the spinal cord, maintains muscle tone and, most importantly, the appropriate tension in the muscles on opposite sides of each joint. Without this functional interconnection, proper joint alignment can't be maintained, and relaxed, upright posture is almost impossible.

Golgi tendon organs are located in the junctions of muscles and their tendons. These protective nerve endings exert a powerful inhibitory effect on contraction of the muscle fibers. They are stimulated by appropriate in proximity stretching of the muscle/tendon junction (as when the muscle fibers are contracting too strongly). Golgi tendon organs transmit their information to the spinal cord and cerebellum through large, rapidly conducting nerve fibers, and they can rapidly inhibit a muscle contraction in order to protect the tendon.

Joint Mechanoreceptors: Surrounding and protecting all joints are tough, fibrous tissues which contain a variety of sensory nerve endings. The input from these specialized sensors keeps the nervous system informed as to the location of the joint and also the degree of stretch, compression, tension, acceleration, and rotation. Multiple simultaneous afferents to a mechanoreceptor facilitate its own efferent, which is the principal purpose of this new design. These joint mechanoreceptors are classified by their anatomy and their neurological function. Type I mechanoreceptors are found in higher densities in the proximal joints. They sense the position of a joint by signaling the joint angle through normal ranges of motion. These help determine postural (tonic) muscle contractions. Type II nerve endings adapt to changes in position, and are most active at onset and termination of movement. These are more densely distributed though the distal joints, and affect phasic muscle actions. Type III mechanoreceptors are high threshold, which means they require considerable joint stress at end ranges before firing. These receptors serve a protective function similar to the Golgi tendon organs. Type IV receptors are free nerve endings located in the ligaments, joint capsules, and articular fat pads which respond with painful stimulus upon extraordinary afferent. They can generate intense, non-adapting motor responses in all muscles related to a joint, resulting in the protective muscle contractions that restrict joint movement.

### PRIOR ART

The earliest percussive technique is often referred to as the hammer-to-the-anvil style, where a mass (the hammer) is set into ballistic motion and acquires a certain speed before it collides with a flat-tipped rod (the anvil) which in turn slaps against the patient. U.S. Pat. No. 4,116,235 (Fuhr et al) and variations of it are examples of this early prior art where the thrust is achieved when an internal spring behind a hammer is compressed until an escapement mechanism releases it. The intent of the hammer-to-the-anvil device is typically to overpower and force joint movement or non-compliant tissue into compliance. The chief disadvantages of this design are that it stings uncomfortably, is limited to one hammer-to-the-anvil slap against tissue per trigger, and has limited adjustment range. Moreover, it does not contemplate preload force. The work that it does is limited to the moment of deceleration when the hammer crashes into the anvil. It is not capable of delivering a continuous train of thrusts and is not sophisticated enough to follow an advanced chiropractic afferent-work-algorithm.

U.S. Pat. No. 7,144,417 (Colloca et al) depicts a more recent implementation of the hammer-to-the-anvil design. In it, instead of a spring, an electrical solenoid propels a ballistic armature (the hammer). Here the intent of a 20 newton preload is to disallow operation at any other preload. Impact force adjustment is via a selector switch, which allows 3 levels of electrical power to the solenoid. This design produces a single hammer-to-the-anvil slap against the tissue, followed by a short train of hammer-to-the-anvil slaps to tissue, said to be 6 Hertz over the interval. The main disadvantages of this design are that it stings uncomfortably and is not capable of delivering a longer continuous train of hammer-to-the-anvil-hits at different pre-selected rates. Furthermore, it is limited to one high hammer speed, one medium hammer speed, or one low hammer speed and one 20 newton preload that if not maintained stops operation. The work that it does is limited to the moment of deceleration when the hammer crashes into the anvil. Colloca is suitable for ballistic—trust only and cannot approach—the ideal and repeatable chiropractic afferent-work-algorithm.

U.S. Pat. Nos. 6,537,236 (Tucek et al) and 6,663,657 (Miller) also achieve the thrusting force via an electrical solenoid, but shun the hammer-to-the-anvil approach. Rather than a hammer abruptly decelerating into an anvil that abuts body tissue; intended here is still inefficient ballistic acceleration, except that the magnetic armature (what amounts to the hammer itself in prior art) directly impacts the body tissue in contact with it. It can still be noisy and uncomfortable but does avoid the instantaneous slap that a crashing anvil creates against the patient's skin. Moving the handle to reposition the armature away from the center of the flux field and then holding that specific position is the method of impact force adjustment. When the armature mass is centered inside the solenoid, there will be minimal axial magnetic force; this is also true when the armature exits the flux field. Thus the chief disadvantage is the method of control is counter-intuitive to the user (forward motion may make the force heavier or lighter) and hence unfriendly. For example: Both designs reposition hammer (armature) force by moving the handle forward towards the patient to increase solid-tissue impact force, except force and travel were not designed to have a direct and reproducible relationship. This is evidenced near mid-travel, where even with the handle moving forward, force behaves unpredictably—thereby creating some confusion in the mind of the practitioner. The majority of practitioners have found this concept unsettling and with limited market appeal, since such therapeutic treatments could not be reliably replicated.

To blunt the end of the rod and guard against pinch point, both Tucek and Miller utilize an often noisy rattling outer spring retained by a nut which is fixed against a linear bearing. After actuation, both Tucek and Miller utilize an inner spring to park the rod against the bumper-stop. In both prior arts, the inner and outer springs lack the defining-characteristics and provide no user-friendly proportion or calibration.

A demand exists for a device that deviates from the commonplace prior art with near ballistic displacement that impacts on bones and non-compliant tissues. Accordingly, it is a general object of the present invention to provide a safe, economical and user friendly device that facilitates a calibrated chiropractic afferent-work-algorithm that is suited to creating multiple mechanoreceptor afferents. A practitioner thus being interactive with this device for applying the algorithm finds how much work/afferentation will feed and re-educate the brain to effect the release of target tissues so they can move without discomfort. These and other objects and advantages of the present invention will be more readily apparent from a consideration of the following drawings and a detailed description of the preferred embodiment.

### SUMMARY OF THE INVENTION

In accordance with one embodiment, the present invention comprises of an algorithmic chiropractic instrument optimized to approximate a chiropractic afferent-work-algorithm for manipulative mechanoreceptor modulation. The present invention is capable of producing a pre-stretch force and power-stretch force that are directly and substantially additive. Unlike any prior art, this new design teaches of an instrument specifically suited to a plunging-probing therapy. The practitioner finds that plunging (manually ramping up the pre-stretch force) acts like a catalyst to steadily increase the instrument's power-stretch output, up to a safety-limit where it plateaus. Further, the practitioner can increase the afferent work done by dwelling at any intermediate pre-stretch level while using feedback from the instrument to probe for a patient-tissue compliance-response. All of this is achieved with a relatively simple physical input through mechanical and electrical implementation, without resorting to the complications of sensor-driven computer electronics. The pre-stretch force is substantially proportional to a tissue like experience over the spine and amplifies tactile, visual, and audio feedback incorporating the patient tissue release response back to the practitioner. The increases of the pre-stretch force by said practitioner is compensated for by a counter-balancing safety limit drop off of the power-stretch.

In accordance with one embodiment, the algorithmic chiropractic instrument further comprises of a lever housing and enclosed within the housing is a reciprocating rod and an electrically energized solenoid having a core mounted in the housing so that the actuator handle part of the housing is longitudinally movable relative to the solenoid. The reciprocating rod comprises of an patient-contact end which can receive numerous therapeutic adapters and a non-magnetic governance ring positioned to critically affect the net magnetic flux distribution of the solenoid.

The reciprocating rod transits through the solenoid and is responsive to the force generated by the solenoid. An inner spring is disposed around the rearward end of the reciprocating rod inside the housing and an outer spring is disposed around the rearward end of the said reciprocating rod outside the housing and compressible. A restricted calibrating adjustment nut is threaded through the rearward end of the reciprocating rod and the position of the adjustment nut governs the tissue pre-stretch upon direct proportional displacement of the inner and outer springs.

The inner spring, outer spring and nonmagnetic ring all acts to algorithmically tether the ballistic nature of solenoid generated power-stretch to produce an efficient and repeatable chiropractic work algorithm, while producing up to 200% the work afferent of prior art.

### BRIEF DESCRIPTION OF THE SEVERAL VIEWS OF THE DRAWINGS

FIG. 1 shows a cutaway view of the present instrument;

FIG. 2 shows the range of the chiropractic afferent-work-algorithm

FIG. 3 shows prior arts' unsafe net force combinations of contact pressure and solenoid force;

FIG. 4 shows protective limiting by the present invention which only allows safe combinations of contact pressure and solenoid force, even with operator error;

FIG. 5 shows the distinction between the present invention and prior art when it comes to the rate of impact against the patient and how the area under the curve defining the available work-range is increased by factor of about 5 by the combination of pre-stretch and tethering of the solenoid;

FIG. 6 shows an algorithmic full range manual probe demonstrating ramp up and safety limiting controls;

FIG. 7 shows a flowchart illustrating interaction between Practitioner and Patient using the present device.

FIG. 8 shows a cutaway view of an alternative embodiment of the present invention.

FIG. 9 shows a cutaway view of an alternative embodiment of the present invention.

### DETAILED DESCRIPTION OF THE PREFERRED EMBODIMENT

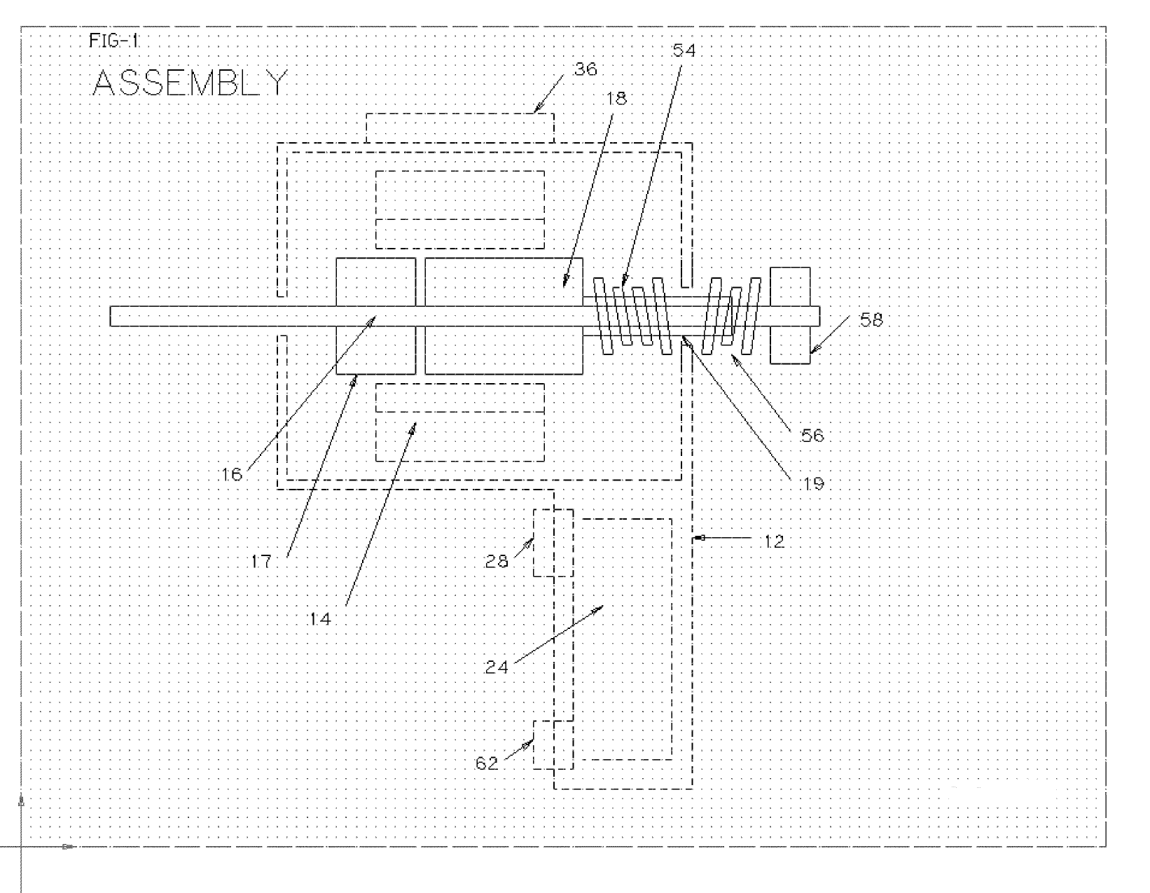

Referring to the drawings, there is depicted a chiropractic instrument in accordance with the present invention, depicted generally in FIG. 1. The instrument in its preferred embodiment comprises a lever housing which is a combination of housing and handle, where the handle is referred to as a hybrid control-lever 12. The lever housing is a critical intermediary and the bio-feedback link between the tactile sense of the practitioner's hand and the palpable compliance of the patient's tissue.

A power-stretch assembly comprising a dual coil solenoid 14 is disposed within the lever housing. The dual coil solenoid 14 provides operational power to the stretch assembly which is further comprised of a reciprocating rod 16 with a patient-contact end disposed at the forward end and secured to the reciprocating rod.

A fan 36 having a specific footprint is provided for airflow to cool the solenoid 14 switches 28, 62 and circuitry 24. Other methods of moving air, including membrane driven pumps may also be utilized.

The reciprocating rod 16 transits through the solenoid 14 and is responsive to a balance point system comprising: the tissue-resistance force, operator pre-stretch force, and the force generated by the dual coil solenoid 14 such that on activation the reciprocating rod 16 is accelerated axially. The reciprocating rod contains a frontward end and rearward end. Fixed on the reciprocating rod 16 is a governance ring 17 and armature 18. An inner spring 54 and an outer spring 56, either side the actuator handle interface, are disposed around the rearward end of the reciprocating rod 16. A threaded adjustment nut 58 is rotatably threaded onto the rearward end of the reciprocating rod 16 to permit adjustment of the balance point of system compression forces of the springs 54, 56. A linear bearing 19 is used to cover and provide a sliding surface to the threading of rod 16. Between stops, the approximate 1 mm thread pitch provides a 4-turn-range of calibrated variate positioning for the armature in the magnetic flux field of the wire coil.

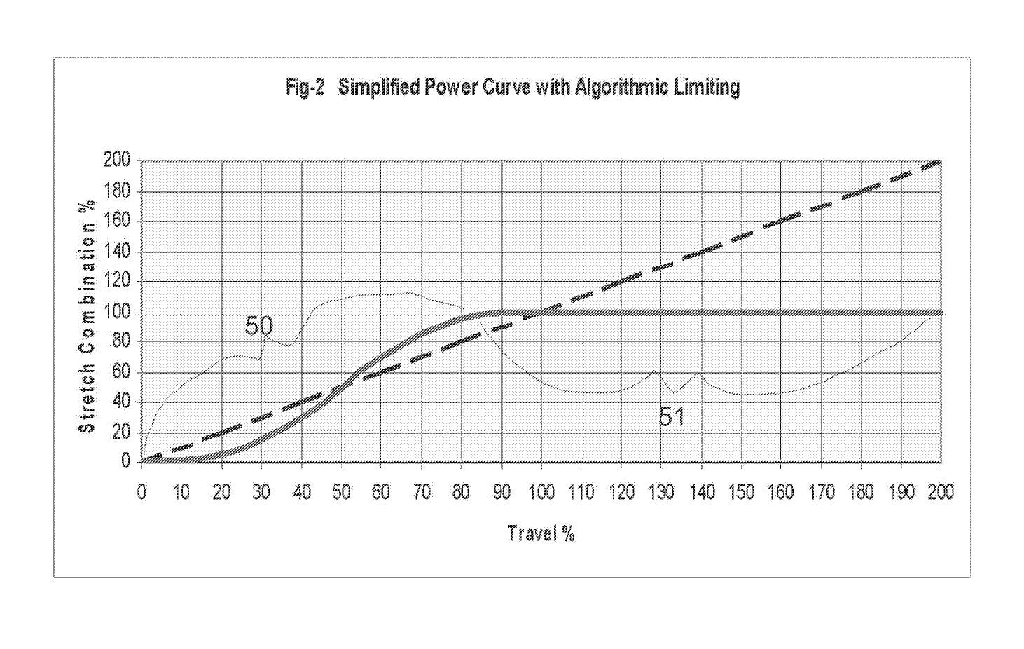

Unlike the ballistic nature of the prior art, the present invention utilizes multiple governance methods to tether the force of the solenoid which accelerates the armature and the reciprocating rod. The present methods of tethering the solenoid 14 is contrary to the teachings of the prior art which insist on maximizing the force of the ballistic solenoid upon impact, which intends to knock bone/hard-tissue into reposition. The present tethering, amplifying, and tactile biofeedback method distinguish the present invention as a device for algorithmically applying a calibrated force that is operator-friendly and can accomplish approximately the chiropractic afferent-work-algorithm of FIG. 2. Thus it transfers waves of power-stretch, cumulatively working mechanoreceptor rich tissues as its substantial pre-stretch sustains the sinking biofeedback layer by layer down to the bone, for greater repositioning. Subject to adjustment nut 58 calibrated compression on outer spring 56, a principal object of inner spring 54 is to provide chiropractor, instrument, and patient interaction. Instrument dynamics follow movements of the hand, which integrates the biofeedback of the variate recoil into a palpable measure of patient response to this afferent-work. By adjusting 58, the practitioner can proportionally find the operate-point for optimum results. In the preferred embodiment, one full turn of the adjustment knob 58 will enable quantified transference of about 25% tissue pre-stretch, 25% of working range for spring 54, 25% of the working force for spring 54, and 25% of the operator afferent modulating force shown in FIG. 2; similar parameter changes at about 50%, 75% and 100%, for turns of 2, 3, and 4, respectively. An object of the inner spring 54 is to equalize operator applied-pressure, patient tissue resistance-pressure, and mechanical solenoid-pressure at a therapeutic tissue stretch level. This creates a patient-operator biofeedback loop in sense perceptible form as the operator, allowed by design, probes synergistically and contributes up to 80% in overall therapeutic afferent work in its preferred embodiment. The inner spring 54, unlike the purely positional springs of the prior art, is an interactively calibrated multi-rate spring with multiple pitch coils, wire size and diameter combination that creates a spring-rate graduation that at a given percentage is proportional to a tissue-like experience over the spine. In the preferred embodiment, the multi-rated spring 54 either has steeper pitch central or decreased diameter central.

The outer spring 56 of the present invention acts as an overpowering governance and amplifies recoil that functions to constantly counter and calibrate the force of spring 54 and the solenoid. In the preferred embodiment, the length of 4 thread pitches exposes 100% of the range which allows the adjustment nut 58 to tighten four whole rotations and visually exposes the length of up to 4 thread pitches as an indication of the combined length of stretch and recoil that is at work. This calculated length of adjustment allows spring 56 to govern the pre-stretch 25% per turn which also allows the practitioner to draw from his past patient history or experience and reproduce prior success upon setting adjustable pre-stretch range the same every time. Once effective, this in practice equates to expediting the algorithm with inner spring 54 contributing to reaching/sinking systematically deeper with governance affecting continuously greater populations of mechanoreceptors within patient tissue as the outer spring 56 recoils which manipulatively does tissue release between solenoid pulses and then re-stretching of mechanoreceptors concentrations at the levels that have already been passed through, due to spring 56's overpowering governance and recoil dissipation action. Additionally, it's this spring-rate proportion governed to suit the chiropractic afferent-work-algorithm that is in itself therapeutic due to the proportional change in force level and nature of its stretch modulation probing and the fact that interactively probing itself contributes to the total work done.

Another novelty of the present invention is the strategic positioning of non-magnetic materials which allows controlled probing of the instrument tip into tissue-under-treatment to achieve slow stretch. The non-magnetic element in the preferred embodiment is depicted as governance ring 17, which is made of aluminum, and works in conjunction with springs 54/56 and the resulting biofeedback pressure-loop to tether the otherwise ballistic force of the solenoid.

The correct use of the above described chiropractic mechanoreceptor modulation instrument results in novel chiropractic afferent-work-algorithm illustrated in FIG. 2 that is significantly different from the prior art. The stretching action of the instrument attributed to the ideal interacting multi-rated springs is seen to be adjustable smoothly and continuously from zero to maximum-permissible over the normal range of possible tissue displacement. The solid line shows actual governed performance and the effect of the tethering methods. In addition to position the desired safety limiting feature, the smooth-control region 50 is governed by a cubic equation, rather than a linear one. This compound curve 50 provides for a slower rate of change with displacement at both the lowest and highest stretch levels. This fine-control provides the practitioner a user-friendliness. Furthermore, approximately 80% of the work curve area depicted in FIG. 2 section 51 is sustained by the therapeutic biofeedback pre-stretch. The therapeutic level of both pre-stretch and power-stretch are directly additive. The pre-stretch being substantially proportional, it serves to induce and expedite resonance compliance. Further, any excess increase of pre-stretch by the practitioner is compensated for by a counter-balancing safety-limit drop off of solenoid power-stretch. This results in a flat curve peak which sustains work level and is what minimizes the effect of achieving power limit with an armature location, which by design is positioned to exit the magnetic flux field proportionally. The flattening of the curve peak is due in part to the algorithmic tethering of the solenoid force. The dashed line presents a purely linear work curve without governing elements such as multi-rated springs.

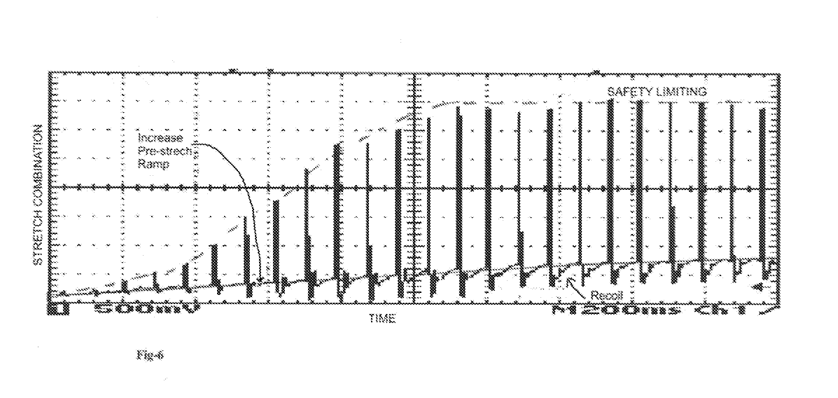

Referring to both FIG. 2 and FIG. 6, the area under the work curve created by the solid line can be viewed as the afferent work done on the patient that is the accumulation of the increments of energy expended during the travel of the instrument tip. Standard equations for work performed is dW=F*dx or alternatively dW=F*v*dt.

Where:

W=work i.e. the integral of either above differentials

F=net applied force

x=displacement distance over which the force is applied

v=velocity of the instrument tip

t=time

Hence the work done on the patient can be viewed as either the area under the curve for tip displacement distance or accumulated time for which the force of the instrument is applied to the patient tissue. For the present invention, each component has an algorithmic function of governing the net force to consistently reproduce the same smooth work curve depicted in FIG. 2. Therefore, the net force may be expressed as:

F=F_{coil}+Fᵢₙₙₑᵣ−Fₒᵤₜₑᵣ−F_{cushion}−F_{friction}−Fₜᵢₛₛᵤₑ−F_{eddy}

Where:
F_{coil}=a function of amperes, armature shape and location
Fᵢₙₙₑᵣ=a function of displacement of the inner spring 54
Fₒᵤₜₑᵣ=a function of displacement of the outer spring 56
F_{cushion}=a function of the elasticity of the clamping bumper-cushion
F_{friction}=a function of the accumulated stylus friction
Fₜᵢₛₛᵤₑ=a function of the elasticity and damped-compliance of patient tissue
F_{eddy}=a function of the eddy current generated by the magnetic field of the solenoid

The present invention's ability to algorithmically tether the blunt force of the solenoid, which in turn allows a slow stretch, which may translate to a higher current and possibly overheating of the solenoid. Therefore, in addition to the fan 36, the present invention also implements a novel coil construction. Typically dual windings solenoid coils have identical turn-counts, e.g. 750 and 750. The present invention utilizes an unbalanced dual coil turn-count which significantly reduces the hot-spot core temperature of the inner winding pushing the heat to the outer winding where it is more readily cooled by convection to the ambient and, in addition, provides for international dual line voltage operation.

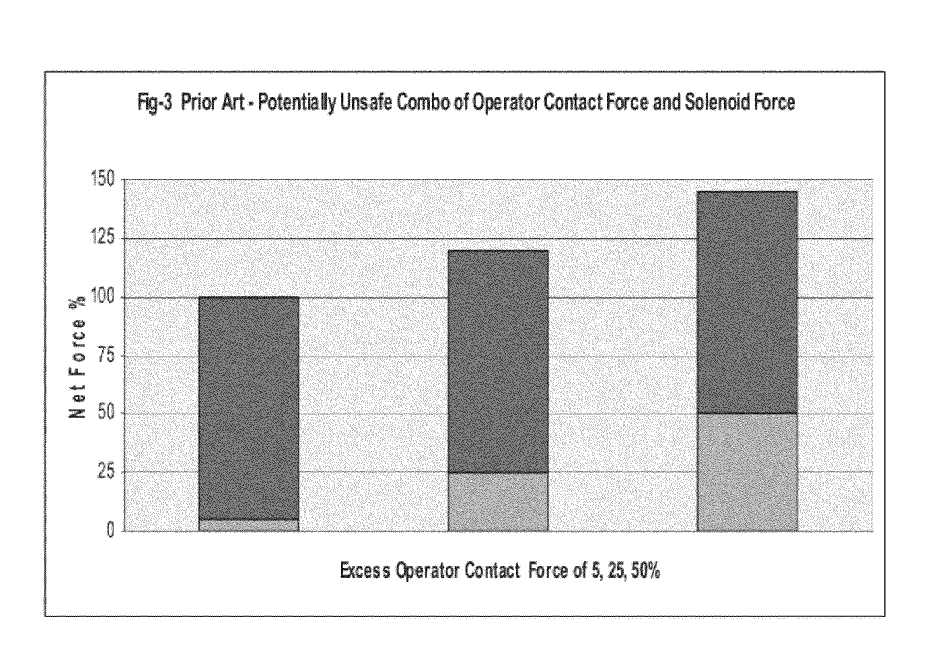

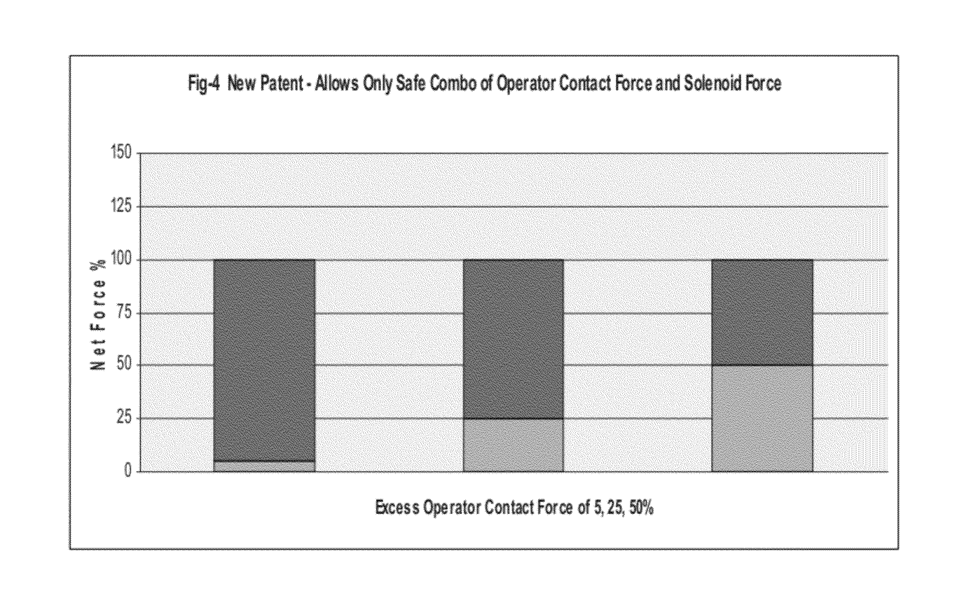

By optimizing armature shape and location in the flux field in conjunction with inner and outer spring parameters, something approximating the ideal algorithmic range of performance of FIG. 2 has been achieved. As the clinical population's average tissue-density, weight, and size changes, it is likewise intended that this algorithm will proportionally change, which recognizes that the health professionals using the instrument are palpators. The way to make the instrument user-friendly for them is to have its tactile sense, audio sense and visual sense perception comparable to the average body tissue displacement density. Ignoring what, in the trade, chiropractors call slack (tissue not likely to be revealing), it was found that a mechanical pre-stretch which approximated the progressive increase in palpable tissue resistance experienced with two-finger-straddling the spinous process was a user friendly proportional-relationship to anyone familiar with palpation. Present day ratio (approximately 4 millimeters at 7 pounds) yields this appreciable perception: that operator's mechanical tactile sensation is relative to human target tissue. Yet when therapy calls for extraordinary displacement, such as an obese patient may require, the machine force against the patient should be safely limited to the instrument rated maximum. Inevitably, due to judgment error, the typical operator instinctively in probing to discern whether the patient requires a level of therapy at or beyond the 100% instrument rating tends to over exert up to about 8 pounds. This can be seen in FIG. 3 where the operator error preload of the prior art devices leads to beyond specification and possibly unsafe levels of force. The preferred algorithm plot recognizes that the act of over-exertion must be virtually sense-perceptible and takes steps in teaching the user not to over exert the instrument beyond its safely limit. FIG. 4 shows the ability of the present algorithmic instrument to stay within 100% of the designated safety limitation of the present device; yet this plateau still allows an up to 200% over-exertion of pre-stretch force.

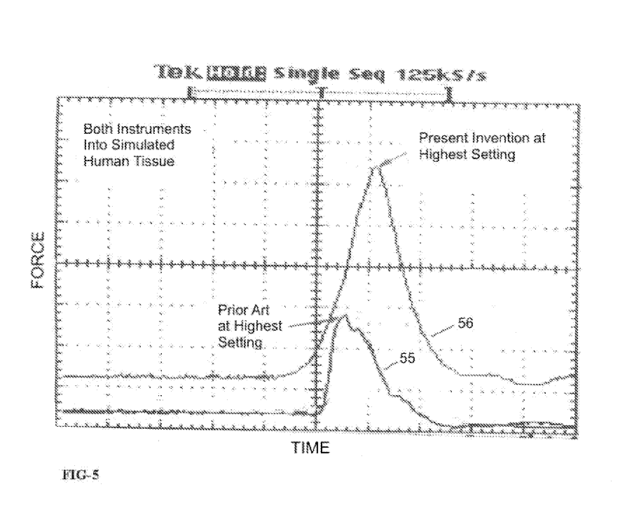

FIG. 5 is an oscilloscope reading of the smooth and efficient impact trust of the present invention vs. that of the more hammer-to-the-anvil impact trust of the prior art. Curve 55 shows a single trust of a prior art device that fails to govern the force of the solenoid. This curve is very ballistic in nature. In contrast, curve 56 shows a single power-stretch generated in part by the solenoid force of the preferred embodiment of the present chiropractic algorithmic instrument and the difference is exceptional. Curve 56 is noticeably smoother, non-ballistic, and can be constantly reproduced.

FIG. 6 shows an oscilloscope reading of multiple power-stretch sequence with algorithmic probing pre-stretch. Notice that the dotted line 61 is substantially similar to the afferent-work-algorithm of a single thrust as seen in the solid line of FIG. 2. The present FIG. 6 further shows the governance method at work in tethering the blunt ballistic nature of the solenoid to produce a smooth work algorithm that peaks despite over-exertion. Another important parameter shown in FIG. 6 is recoil. Each safely limited positive power-stretch of the instrument is immediately followed by a recoil thrust in the opposite direction, though still a net-positive due to the initial pre-stretch force. This variate recoil is one of the sine qua non of this invention. It provides the practitioner with an amplified tactile and audio feedback which incorporates a direct measure of tissue-release in the patient. This variate recoil is chiefly the result of the complex and collaborative tethering action of the inner spring 54, the Lenz-force due to eddy-current in ring 17, the threshold set by the initial adjustment of outer spring 56 and balancing practitioner-pre-stretch against the magnetic force of the triggered solenoid. The audio feedback, i.e. the sound created by the spring recoil after each power-stretch, is also algorithmic and unique. As the pre-stretch force changes, the sound pitch changes synchronously, thus providing consistent feedback from patient to practitioner. By design as the instrument exceeds maximum pre-stretch, a distinct sound is produced.

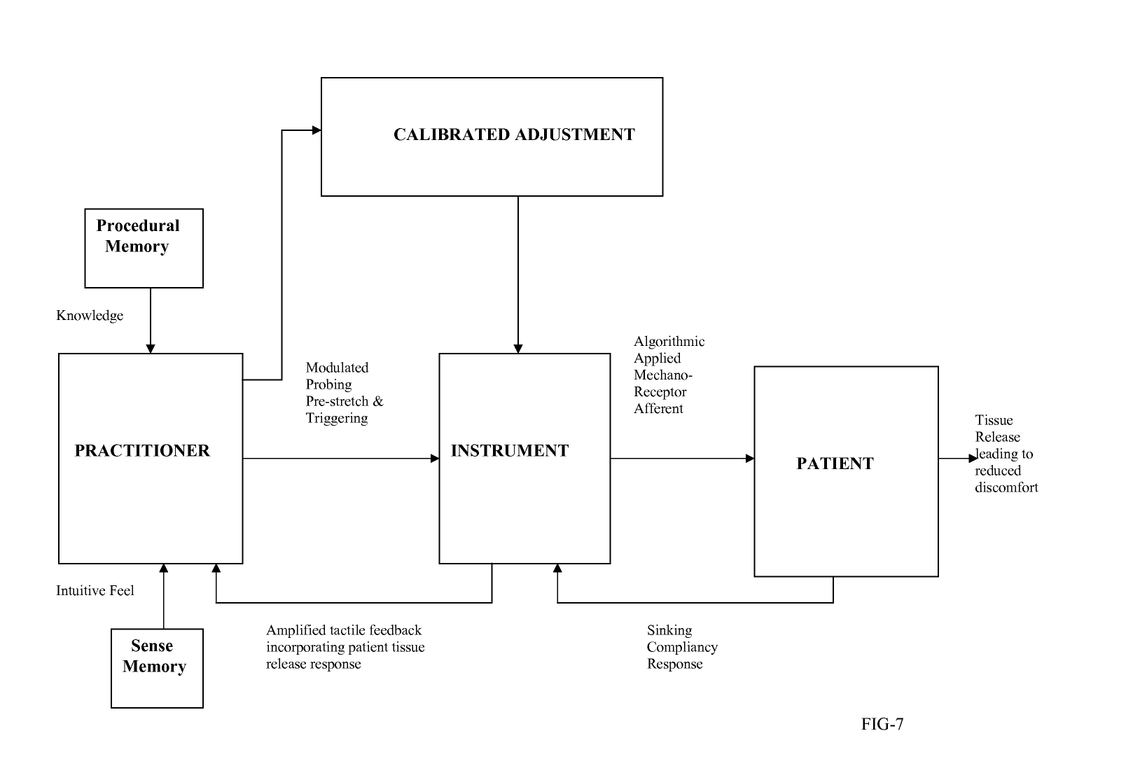

FIG. 7 shows the flowchart of the patient-operator biofeedback loop focusing on the functions of the practitioner, the preferred embodiment of the chiropractic instrument and the patient. The practitioner while drawing from procedural memory and sensory memory applies initial force to the lever housing based on his training, but then adjusts it in a stepwise manner based on this tactilely and audibly sensed feedback from the instrument. The practitioner also has control over the calibrated adjustment of the instrument which sets the initial threshold for pre-stretch force and produces a variate amplified tactile feedback from the instrument within its range. The instrument controlled by the practitioner applies the afferent work algorithmic force. This calculated and non-ballistic force allows the patient's brain to reduce resistance to change which leads to tissue release. While applying the chiropractic instrument to the patient, the substantially proportional pre-stretch ratios induce resonance compliance, and the instrument transfers and amplifies tactile feedback incorporating the patient tissue release response back to the practitioner so that once again the practitioner can adjust his own force modulated probing and force according to procedural and sensory memory.

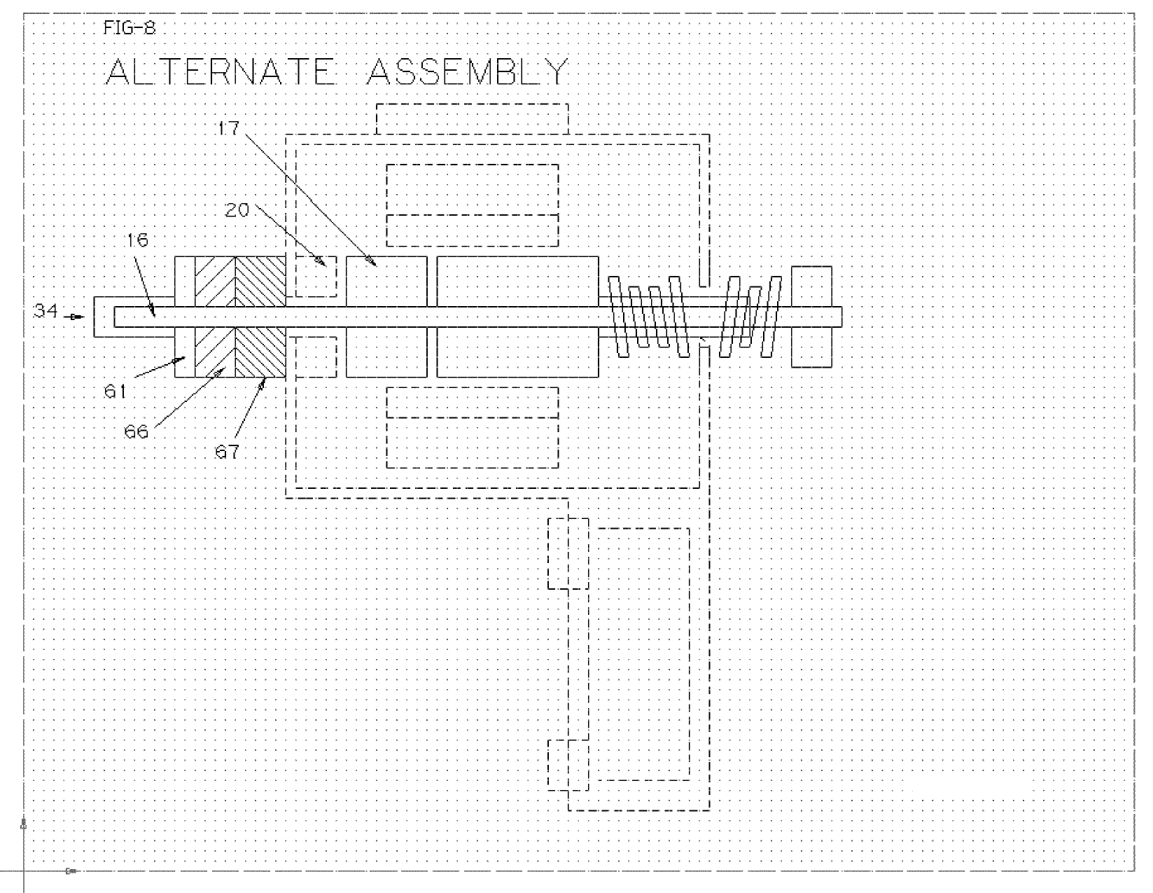

FIG. 8 depicts an alternative embodiment where the reciprocating rod 16 further comprises of a collar 61, and the housing further comprise of collar-stop and bumpers 20. The non-magnetic governance ring 17 acts as a bumper stop wherein the reciprocating rod's travel length is limited to the space between ring 17 and bumper 20. The collar 61 also limits the pre-stretch maximum because once collar 61 contacts the outside of the housing, the maximum allowable pre-stretch is reached. The setting of adjustment nut 58 would then govern the position of the collar 61 and ring 17 in conjunction with the housing bumpers 20. Furthermore, the positions of both collar 61 and ring 17 may be manually and independently altered to approximate or mirror image the pre-stretch concept at the forward-end of the rod to effect a substantially additive variate of pre-stretch, perhaps as layered densities of a foam-cushion-stop depicted as toroids 66 and 67, which is effectively just an alternate embodiment of multi-pitch springs 54 and 56.

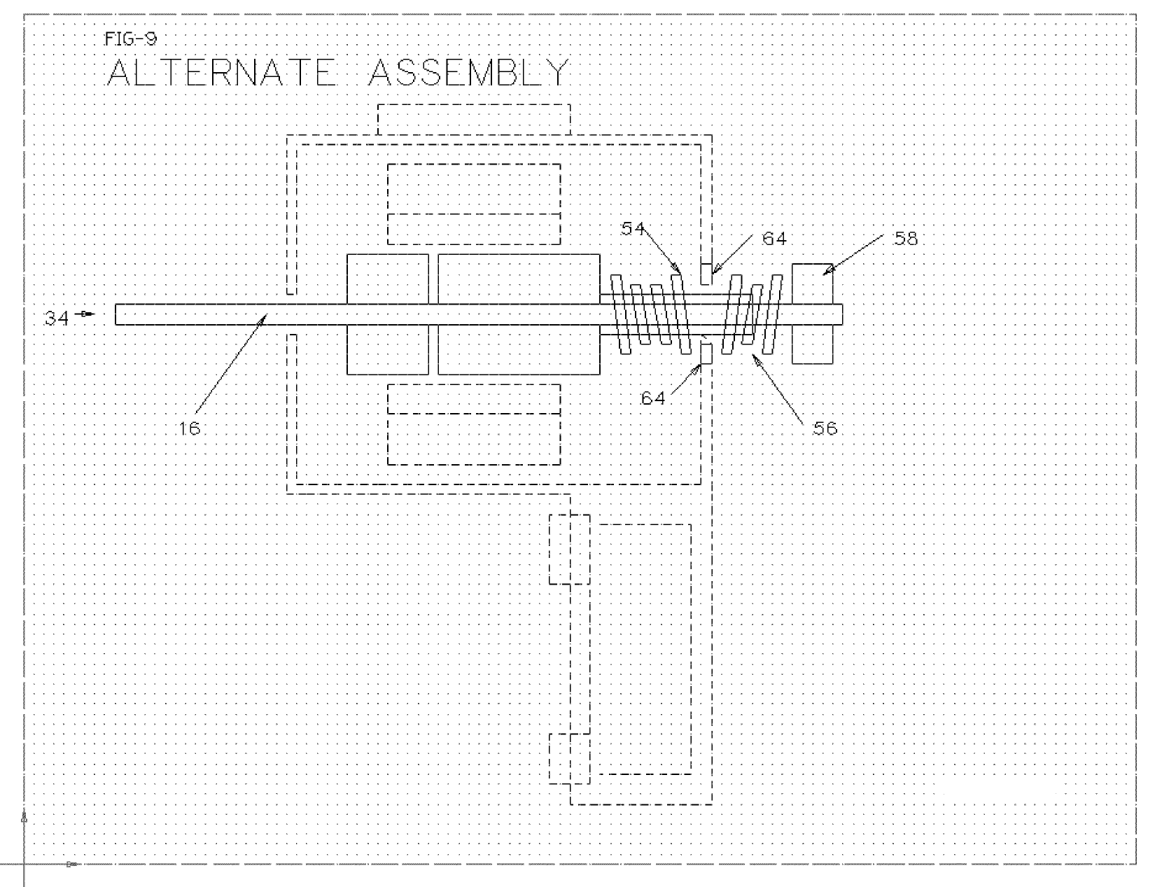

In another alternative embodiment, a novel switch integration illustrated in FIG. 9, searches at the balance point of forces, effectively floats the threshold of switching action to be synchronous with the set balance-point determined by the adjusting knob 58. This switch in conjunction with knob 58 makes it more convenient for the practitioner to draw from his past patient history or experience and reproduce the prior therapy level of success upon setting adjustable pre-stretch range noted in the patient's record and may be implemented to train biofeedback and consistency among operators relative to tissue stretch as operators contact patients with pre-stretch equal to the compression on spring 54. More specifically, when the extended patient-contact end 34 is displaced coaxially into the body of the patient and the inner-spring 54 is compressed against the inside of the plastic handle in proportion to the pre-stretch. The outer-spring 56 is decompressed relative to the pre-stretch as it releases away from the handle by the action of transferring pre-stretch (otherwise known as a particular prior reproducible point on the algorithm). Among other things, the amount of decompression of 56 is a function of its original adjustment position along the stylus rod 16. For a given adjustment position, when 56 is secured to the nut 58, the outer-spring 56 will gap away from the handle surface at a specific magnitude of pre-stretch. A pair of metallic contacts 64 added to the handle in conjunction with the metal of the outer-spring 56 is the preferred way to detect the point of balance or transference the precise occurrence of this pre-stretch induced gap. The simplest pre-stretch sensing contact configuration is a normally-closed electrical pair. The instrument trigger-switch is typically a normally-open contact pair, so an inversion is usually required. This can be easily achieved with an electro-mechanical relay or some other conventional electronic circuit technique. The inverted-pair can then be used in parallel with the trigger to cause actuation in conjunction with the setting of adjustment nut 58, which thus allows matching to a particular point on the chiropractic afferent-work-algorithm.

In yet another embodiment, the reciprocating rod 16, or strategic portions of it, can be made of either magnetic or non-magnetic material so as to allow for a therapeutic magnetic flux field at the patient interface.

Throughout the specification the aim has been to describe the invention without limiting the invention to any one embodiment or specific collection of features. People of ordinary skill in the relevant art may realize variations from the specific embodiment that will nonetheless fall within the scope of the invention. For example the means of tethering the solenoid is not limited to elements disclosed in the preferred embodiment, the springs 54 and 56 is not limited to any specific tension or shape; they may have electromagnetic, elastic, pneumatic, or hydraulic buffering. The non-ferromagnetic ring 17 can be of any material and could be located anywhere within the housing as long as it affects the magnetic flux force of the solenoid to have proportional interaction.

The total work done by the chiropractic afferent-work-algorithm of the present invention is not limited as the invention may apply for athletes, an up-sized instrument having the same afferent-work-algorithm proportionality but perhaps with a nameplate rating at approximately 50% higher level of mechanoreceptor modulation energy. This then would require a more powerful winding and dynamically tuned governing spring characteristics and approximately 50% increase for maximum preload. Despite the size increase of the instrument itself, the chiropractic work algorithm of the present invention still proportionally exists.

Accordingly, the scope of the invention should be determined not by the embodiment(s) illustrated, but by the appended claims and their legal equivalents.

I claim:

1. An algorithmic chiropractic instrument optimized to approximate a chiropractic afferent-work-algorithm for manipulative mechanoreceptor modulation capable of
a. producing a pre-stretch force and a power-stretch force that are directly and substantially additive;
b. the said pre-stretch force is substantially proportional to a tissue like experience over the spine and amplifies tactile, visual, and audio feedback incorporating the patient tissue release response back to the practitioner; and
c. wherein the increases of said pre-stretch by said practitioner is compensated for by a counter-balancing safety limit drop off of said power-stretch.

2. An algorithmic chiropractic instrument of claim 1 optimized to approximate a chiropractic afferent-work-algorithm for a proportional plunging-probing style of manipulative mechanoreceptor modulation and provides biofeedback to the practitioner.

3. An algorithmic chiropractic instrument of claim 1 further comprising:
a. a lever housing;
b. a reciprocating rod having an patient-contact end and mechanical stops to limit axial travel of the rod in either direction;
c. an electrically energized solenoid mounted in the said lever housing so that the said lever housing is longitudinally movable independent of said solenoid;
d. said reciprocating rod comprising of a non-magnetic governance ring, a armature, threading, and a linear bearing;
e. said reciprocating rod transits through the solenoid and is responsive to the force generated by said solenoid;
f. an inner spring disposed around the rearward end of the said reciprocating rod inside said lever housing;
g. an outer spring disposed around the rearward end of the said reciprocating rod outside said housing and compressible;
h. a restricted calibrating adjustment nut threaded through the rearward end of said reciprocating rod wherein said adjustment nut governs the human tissue pre-stretch co-dependently with said inner and outer springs; and wherein
i. said inner spring, outer spring and nonmagnetic ring's position in the magnetic flux field act within the range of the mechanical stops to algorithmically tether the ballistic nature of said solenoid to produce an efficient and repeatable chiropractic work algorithm which intentionally and uniquely combines substantially intended to be additive pre-stretch and power-stretch.

4. The algorithmic chiropractic instrument of claim 3 wherein the said inner spring contributes to reaching and sinking into said mechanoreceptors on a patient while said outer spring contributes to tissue stretching and re-stretching of said mechanoreceptors of said patient.

5. The algorithmic chiropractic instrument of claim 4 wherein the said chiropractic work algorithm comprises first of a compound curve attributed to the governance of both said inner and outer springs and then leading to a flattening peak attributed to said tethering of the force of said solenoid and additive pre-stretch.

6. The algorithmic chiropractic instrument of claim 5 wherein twenty-to-eighty percent of the work done in the afferent work algorithm is sustained by said pre-stretch force substantially within therapeutic level generated by said practitioner and said inner and outer springs.

7. The algorithmic chiropractic instrument of claim 5 wherein said inner spring and said outer springs are multi-rated and said inner spring is a variate tethering of said solenoid relative to percent of the work length and said outer spring provides overpowering governance of said solenoid force.

8. The algorithmic chiropractic instrument of claim 7 wherein the said work algorithm can substantially replicate a net force by balancing the coil force, inner spring force, outer spring force, cushion force, friction force, tissue force, and eddy current force.

9. The algorithmic chiropractic instrument of claim 5 wherein the tethering of the said solenoid prohibits said instrument to exceed force of designated safety limit.

10. The algorithmic chiropractic instrument of claim 5 wherein said outer spring is selected to amplify recoil force against the power stretch force created by said solenoid for the purpose of improving tactile feedback as a function of said pre-stretch force.

11. A Chiropractic instrument of claim 5 wherein the said instrument provides a power-stretch and relays biofeedback and is reproducible.

12. The algorithmic chiropractic instrument of claim 3 wherein the said inner spring has a spring-rate graduation that at a given percentage is proportional to a tissue like experience over the spine for the average person.

13. The algorithmic instrument of claim 12 wherein:
a. the said inner and outer springs, although purposely different in size or force or rate, result in equal-and-opposite force on said lever-housing;
b. said inner and outer springs respond predictably and algorithmically thus providing a continuous and physically interactive interface between practitioner-stimulus and patient-response; and
c. produces a steady bio-feedback that informs the practitioner as to the efficacy of his continual adjustments and produces a controlled stepwise therapy and an algorithmic process.

14. The algorithmic chiropractic instrument of claim 3 wherein the said nonmagnetic ring can be located anywhere within the said housing as long as it appropriately affects the magnetic flux of the said solenoid.

15. The algorithmic chiropractic instrument of claim 14 wherein the said nonmagnetic ring comprises of aluminum.

16. The algorithmic chiropractic instrument of claim 3 wherein a separate integral switch contact actuates said instrument when electrical circuit continuity is broken coincident with an adjustable pre-stretch force threshold having been achieved by the operator and a gap opens between said outer spring and electrical contacts on said lever housing.

17. The algorithmic chiropractic instrument of claim 3 wherein the said solenoid comprises of unbalanced dual coil turn-counts.

18. An algorithmic chiropractic instrument of claim 3 wherein said pre-stretch is enough to induce resonance compliance with said power-stretch

19. A chiropractic instrument of claim 3 that provides tactile measures, both direct visual and mechanical turn-number, as calibration status.

20. A chiropractic instrument of claim 3 that provides a scale-factor for calibration where each adjustment-turn of said adjustment nut proportionally adjust said pre-stretch, work range of said inner spring, work force of said inner spring, and afferent modulating force of said practitioner.

21. A chiropractic instrument of claim 3 further comprising:
a. a collar located on said reciprocating rod outside of said housing; and
b. a bumper located inside the housing capable of limiting the travel range of said reciprocal rod.

22. A method for applying a chiropractic afferent-work-algorithm comprising:
a. calibrating initial threshold for pre-stretch force on a reciprocating chiropractic instrument that would produce an amplified tactile feedback from said instrument;
b. modulated probing of a patient with said reciprocating chiropractic instrument wherein said reciprocating chiropractic instrument contains a means of creating a patient-operator biofeedback loop; and
c. triggering said reciprocating chiropractic instrument upon said patient whereby applying a systematic afferent stimulation via an algorithmic barrage of stretches wherein said barrage of stretches is an algorithmic applied force.

23. An approximate chiropractic afferent-work-algorithm created by an algorithmic chiropractic instrument comprising:
a. a smooth control work region with a slower rate of change with displacement at both the lowest and highest stretch levels, and
b. a flattening curve peak work region due to algorithmic tethering of the solenoid force.

24. An approximate chiropractic afferent-work-algorithm created by an algorithmic chiropractic instrument of claim 23 wherein twenty-to-eighty percent of the substantially additive work of said afferent work algorithm is sustained by the therapeutic biofeedback pre-stretch of said algorithmic chiropractic instrument.

25. An approximate chiropractic afferent-work-algorithm created by an algorithmic chiropractic instrument of claim 23 wherein said smooth control work force region and said flattening curve peak work region are results of algorithmic balancing of a dual spring and nonmagnetic material balance point system that balances the tissue resistance force, operator pre-stretch force, and said solenoid force.

26. An approximate chiropractic afferent-work-algorithm created by an algorithmic chiropractic instrument of claim 23 wherein the said instrument provides a reproducible power-stretch and relays biofeedback during said reproducible power-stretch.
